(19) United States
(12) Patent Application Publication
(10) Pub. No.: US 2015/0224086 A1
(43) Pub. Date: Aug. 13, 2015

(54) Pharmaceutical Formulations of Rufinamide

(71) Applicant: Hetero Research Foundation, Andhra Pradesh (IN)

(72) Inventors: Bandi Parthasaradhi Reddy, Andhra Pradesh (IN); Podili Khadgapathi, Andhra Pradesh (IN); Goli Kamalakar Reddy, Andhra Pradesh (IN)

(73) Assignee: Hetero Research Foundation, Andhra Pradesh (IN)

(21) Appl. No.: 14/422,884

(22) PCT Filed: Aug. 5, 2013
(86) PCT No.: PCT/IN2013/000482

§ 371 Date: Feb. 20, 2015

(30) Foreign Application Priority Data
Aug. 23, 2012 (IN) 3466/CHE/2012

(51) Int. Cl. A61K 31/4192; A61K 47/32

(52) U.S. Cl. CPC A61K 31/4192; A61K 47/32

(57) ABSTRACT

The present invention relates to pharmaceutical compositions comprising rufmamide premix and one or more pharmaceutically acceptable excipients and methods of preparing the same.

### PRIORITY

This patent application claims priority to Indian patent application number 3466/CHE/2012, filed on Aug. 23, 2012, the contents of which are incorporated by reference herein in their entirety.

### FIELD OF THE INVENTION

The present invention relates to pharmaceutical compositions comprising rufinamide premix and one or more pharmaceutically acceptable excipients and methods of preparing the same.

### BACKGROUND OF THE INVENTION

Chemically rufinamide is 1-[(2,6-difluorophenyl)methyl]-1H1,2,3-triazole-4 carboxamide. It has a molecular formula C₁₀H₈F₂N₄O, molecular weight of 238.2, and structural Formula I.

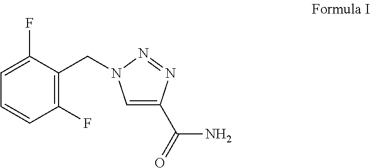 [SMILES: NC(=O)c1cn(Cc2c(F)cccc2F)nn1]

Commercially available formulation containing rufinamide is sold by Eisai, under the brand name of BANZEL®, in the form of 200 mg, 400 mg oral tablets and 40 mg/ml oral suspension. BANZEL® is indicated for adjunctive treatment of seizures associated with Lennox-Gastaut syndrome.

U.S. Pat. No. 4,789,680 disclose rufinamide.

U.S. Pat. No. 8,076,362 disclose tablet composition of rufinamide crystal modification A with microcrystalline cellulose.

U.S. Pat. No. 6,455,556 disclose rufinamide crystal modification B and C.

An unpublished Patent Application No. IN 2972/CHE/2012 disclose solid dispersion of rufinamide.

There remains a need to develop pharmaceutical compositions comprising solid dispersion of rufinamide. Accordingly, inventors of the present invention have developed compositions of rufinamide premix that were found to be comparable with marketed BANZEL® tablets.

### SUMMARY OF THE INVENTION

The present invention provides pharmaceutical compositions comprising rufinamide premix and one or more pharmaceutically acceptable excipients and process for their preparation.

In embodiment, the present invention includes pharmaceutical composition comprising rufinamide premix, crospovidone as superdisintegrant and one or more pharmaceutically acceptable excipients.

In an embodiment, the present invention includes pharmaceutical tablet composition comprising rufinamide premix as an active agent, crospovidone as superdisintegrant and one or more excipients selected from lactose, sodium lauryl sulphate, colloidal silicon dioxide and magnesium stearate.

In an aspect, the present invention provides process for preparing compositions of rufinamide premix by wet granulation involving: i) sifting and blending rufinamide premix optionally with one or more pharmaceutically acceptable excipients to form a dry blend, ii) granulating the dry blend of step (i), using a solvent or binder solution containing sodium lauryl sulphate, followed by drying and sizing to get desired granules, iii) blending the granules of step (ii) with at least one pharmaceutically acceptable excipient, iv) lubricating the granules of step (iii), and finally compressing into tablets.

In another aspect, the pharmaceutical composition comprising therapeutically effective amount of rufinamide is indicated for adjunctive treatment of seizures associated with Lennox-Gastaut syndrome.

### DETAILED DESCRIPTION OF THE INVENTION

The present invention discloses pharmaceutical compositions comprising rufinamide premix and one or more pharmaceutically acceptable excipients and process for preparing the same.

The term “active ingredient” herein refers to a pharmaceutically active molecule as well as its pharmaceutically acceptable and therapeutically active salts, esters, amides, prodrugs, metabolites, enantiomers, polymorphs, analogs, etc. that induce a desired pharmacological or physiological effect. Terms like “active”, “active agent”, “active substance” may be used synonymously for “active ingredient”.

The term “pharmaceutically acceptable” as used herein means that which is useful in preparing a pharmaceutical composition that is generally non-toxic and is not biologically undesirable and which is acceptable for veterinary use and/or human pharmaceutical use.

The term “excipients” as used herein means a component of a pharmaceutical product that is not an active ingredient such as, for example, fillers, diluents, carriers and the like. The excipients that are useful in preparing a pharmaceutical composition are generally safe, non-toxic and are acceptable for veterinary use as well as human pharmaceutical use.

The term “composition” or “formulation” or “dosage form” as used herein refers to a solid dosage form suitable for administration, such as a tablet, capsule, granules, pill, etc.

The term “therapeutically effective amount” means the amount of an active agent that is sufficient to treat or prevent the disease. The “therapeutically effective amount” will vary depending on the active agent, the disease and its severity and the age, weight, etc., of the patient to be treated.

As used in this specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

Rufinamide premix according to the present invention comprises solid dispersion of rufinamide prepared as per the disclosure of IN 2972/CHE/2012 assigned to Hetero Research Foundation.

Solid dispersion of rufinamide according to the present invention comprises one or more pharmaceutically acceptable excipients selected from hydroxypropyl methylcellulose, copovidone, sorbitan monolaurate (span 20), ethyl cellulose, polyethylene glycol and a graft copolymer comprised of polyethylene glycol, polyvinylcaprolactam and polyvinylacetate (soluplus) or a combination thereof.

The present invention describes selection of a superdisintegrant for preparing the pharmaceutical compositions of rufinamide premix with desired dissolution profile.

Accordingly inventors of the present invention have prepared rufinamide premix compositions using different superdisintegrants like crospovidone, croscarmellose sodium and sodium starch glycolate. Such compositions were evaluated for disintegration time and in vitro dissolution time to determine the rate and extent at which the active substance is released from the dosage forms. Compositions containing crospovidone as superdisintegrant showed excellent disintegration and dissolution properties as compared to compositions with croscarmellose sodium or sodium starch glycolate. This indicates that the dissolution of dosage forms of rufinamide premix can be improved by using crospovidone as superdisintegrant.

Crospovidone according to the present invention is present either extragranularly, or intragranularly or both in an amount of 1 to 15%, preferably 3 to 12% based on total weight of the composition.

In one aspect, pharmaceutical composition comprises rufinamide premix, crospovidone as superdisintegrant and one or more pharmaceutically acceptable excipients.

Useful pharmaceutically acceptable excipients are those known to persons skilled in the art and may include, but are not limited to, any one or more of diluents, binders, surfactants, glidants and lubricants.

Exemplary diluents include but are not limited to lactose, dibasic calcium phosphate, tribasic calcium phosphate, calcium carbonate, calcium sulfate, magnesium carbonate, magnesium oxide, talc, sugar, starches, mannitol, sorbitol, inorganic salts, cellulose derivatives, calcium sulfate, xylitol, lactitol, kaolin, sucrose, dextrates, dextrin, maltodextrin, dextrose and the like, and combinations thereof.

Exemplary binders include but are not limited to polyvinyl pyrrolidone, hydroxypropyl cellulose, hydroxypropyl methylcellulose, powdered acacia, gelatin, guar gum, carbomers and the like, and combinations thereof.

Exemplary surfactants include but are not limited to sodium lauryl sulphate, soluplus (a graft copolymer comprised of polyethylene glycol, polyvinylcaprolactam and polyvinylacetate), polyoxyethylene-polyoxypropylene block copolymers (also known as poloxamers), polyethylene glycols, sodium stearyl sulfate, sodium oleyl sulfate, sodium cetyl sulfate, sodium dodecylbenzene sulfonate, dialkyl sodium sulfosuccinates, polysorbates and the like, and combinations thereof.

Exemplary glidants include but are not limited to colloidal silicon dioxide, calcium silicate, magnesium silicate, silicon hydrogel, cornstarch, talc and the like; and combinations thereof.

Exemplary lubricants include but are not limited to magnesium stearate, calcium stearate, zinc stearate, stearic acid, fumaric acid, palmitic acid, talc, sodium stearyl fumarate, carnauba wax, hydrogenated vegetable oils, mineral oil, polyethylene glycols and the like, and combinations thereof.

In one aspect, pharmaceutical tablet composition comprises rufinamide premix as an active agent, crospovidone as superdisintegrant and one or more excipients selected from lactose, sodium lauryl sulphate, colloidal silicon dioxide and magnesium stearate.

In another aspect, the present invention relates to pharmaceutical tablet composition comprising based on total weight of the composition, i) 50 to 90 wt % of rufinamide premix as an active agent, ii) 1 to 15 wt %, preferably 3 to 12% of crospovidone as superdisintegrant and iii) one or more excipients selected from lactose, sodium lauryl sulphate, colloidal silicon dioxide and magnesium stearate.

Equipments suitable for processing the pharmaceutical formulations include one or more of mechanical sifters, granulators, blenders, compression machines, fluid bed processors, etc.

The pharmaceutical formulations of rufinamide premix may be processed by either wet granulation, dry granulation or by direct compression comprising one or more pharmaceutically acceptable exicipients.

In particular, the present disclosure relates to wet granulation processes for preparing solid dosage forms comprising rufinamide premix and one or more pharmaceutically acceptable excipients.

Accordingly, the present invention provides process for preparing compositions of rufinamide premix by wet granulation involving: i) sifting and blending rufinamide premix optionally with one or more pharmaceutically acceptable excipients to form a dry blend, ii) granulating the dry blend of step (i), using a solvent or binder solution containing sodium lauryl sulphate, followed by drying and sizing to get the desired granules, iii) blending the granules of step (ii) with at least one pharmaceutically acceptable excipient, iv) lubricating the granules of step (iii), and finally compressing into tablets.

The present invention further relates to pharmaceutical composition of rufinamide premix with crospovidone and one or more excipients, prepared by wet granulation process.

A film coat on the tablet provides an elegant appearance and further contributes to the ease with which it can be swallowed.

Pharmaceutical compositions of the present invention comprising therapeutically effective amount of rufinamide is indicated for adjunctive treatment of seizures associated with Lennox-Gastaut syndrome.

### EXAMPLES

Certain specific aspects and embodiments of this invention are described in further detail by the examples below, which are provided only for purposes of illustration and are not intended to limit the scope of the invention in any manner.

### Example 1-3

Tablet compositions comprising Rufinamide premix prepared by wet granulation method:

TABLE 1

| | Example-1 | Example-2 | Example-3 |
|---|---|---|---|
| Ingredient | mg/unit | mg/unit | mg/unit |
| Dry mix | | | |
| Rufinamide premix^{#} | 1120.00 | 1120.00 | 1120.00 |
| Wet granulation | | | |
| Sodium lauryl sulphate | 15.00 | 15.00 | 15.00 |
| Purified water | q.s. | q.s | q.s |
| Extra-granular ingredients | | | |
| Lactose monohydrate | 135.00 | 135.00 | 135.00 |
| Crospovidone | 100.00 | — | — |
| Croscarmellose sodium | — | 100.00 | — |
| Sodium starch glycolate | — | — | 100.00 |
| Colloidal silocon dioxide | 20.00 | 20.00 | 20.00 |
| Lubrication | | | |
| Magnesium stearate | 10.00 | 10.00 | 10.00 |
| Film coating | | | |
| Opadry ® pink | 28.00 | 28.00 | 28.00 |
| Purified water | q.s | q.s | q.s |
| Film coated tablet weight | 1428.00 | 1428.00 | 1428.00 |

^{#}Each 1120 mg of Rufinamide premix contains 400 mg of Rufinamide.

### Manufacturing Process for Examples 1 to 3:

1. Rufinamide premix was sifted through mesh #30 sieve and dry blended for 10 minutes,
2. granulating fluid was prepared using sodium lauryl sulphate and purified water,
3. blend of step 1 was granulated using granulating fluid of step 2, followed by drying and sifting to get the desired granules,
4. extra-granular ingredients were sifted through mesh #40 sieve,
5. granules of step 3 were blended with sifted materials of step 4,
6. magnesium stearate was sifted through mesh #60 sieve,
7. granules of step 5 were lubricated with magnesium stearate of step 6,
8. lubricated granules of step 7 were compressed into tablets,
9. tablets of step 8 were film coated using an Opadry® pink dispersion.

### Comparative Study on Disintegration Time:

The film coated tablets obtained in Examples 1-3 were evaluated for disintegration time as shown in Table 2.

TABLE 2

| | Disintegration time |
|---|---|
| Example 1 | 13 minutes |
| Example 2 | 25 minutes |
| Example 3 | 29 minutes |

Based on results presented in Table 2, Example-1 containing crospovidone as superdisintegrant showed good disintegration properties when compared to Example 2 and 3 containing croscarmellose sodium and sodium starch glycolate respectively.

### Comparative Study on Dissolution Time:

Dissolution Medium: 6.8 pH phosphate buffer+2% w/w sodium lauryl sulphate

### Volume: 2000 ml

### Apparatus: USP II (Paddle)

### Speed: 100 RPM

TABLE 3

| Capsule con- | | | | | | |
|---|---|---|---|---|---|---|
| taining 400 mg | % drug release at different time intervals | | | | | |
| of rufinamide | 15 min | 30 min | 45 min | 60 min | 90 min | 120 min |
| Banzel | 50 | 65 | 69 | 74 | 76 | 80 |
| (Reference) | | | | | | |
| Example-1 | 46 | 77 | 87 | 91 | 95 | 95 |
| Example-2 | 23 | 56 | 69 | 73 | 80 | 81 |
| Example-3 | 21 | 52 | 65 | 71 | 75 | 79 |

As can be seen from Table 3, Example-1 containing crospovidone as superdisintegrant showed excellent dissolution properties as compared to Examples 2 and 3. Thus, the data in Table 3 indicates that the dissolution of dosage forms of rufinamide premix can be improved by using crospovidone as superdisintegrant.

### Example 4

Tablet compositions comprising Rufinamide premix prepared by wet granulation method:

| Ingredient | mg/unit |
|---|---|
| Dry mix | |
| Rufinamide premix^{@} | 560.00 |
| Wet granulation | |
| Sodium lauryl sulphate | 7.5 |
| Purified water | q.s. |
| Extra-granular ingredients | |
| Lactose monohydrate | 67.50 |
| Crospovidone | 50.00 |
| Colloidal silocon dioxide | 10.00 |
| Lubrication | |
| Magnesium stearate | 5.00 |
| Film coating | |
| Opadry ® pink | 14.00 |
| Purified water | q.s |
| Film coated tablet weight | 714.00 |

^{@}Each 560 mg of Rufinamide premix contains 200 mg of Rufinamide.

### Manufacturing Process: Same as Example 1.

### Example 5-7

Tablet compositions comprising Rufinamide premix prepared by wet granulation method:

| | Example-5 | Example-6 | Example-7 |
|---|---|---|---|
| Ingredient | mg/unit | mg/unit | mg/unit |
| Dry mix | | | |
| Rufinamide premix | 1000.00* | 1200.00** | 800.00*** |
| Wet granulation | | | |
| Sodium lauryl sulphate | 15.00 | 20.00 | 15.00 |
| Purified water | q.s. | q.s. | q.s. |
| Extra-granular ingredients | | | |
| Lactose monohydrate | 205.00 | 85.00 | 300.00 |
| Crospovidone | 100.00 | 65.00 | 55.00 |
| Colloidal silocon dioxide | 20.00 | 20.00 | 20.00 |
| Lubrication | | | |
| Magnesium stearate | 10.00 | 10.00 | 10.00 |
| Film coating | | | |
| Opadry ® pink | 27.00 | 28.00 | 24.00 |
| Purified water | q.s | q.s | q.s |
| Film coated tablet weight | 1377.00 | 1428.00 | 1224.00 |

*Each 1000 mg of Rufinamide premix contains 400 mg of Rufinamide.
**Each 1200 mg of Rufinamide premix contains 400 mg of Rufinamide.
***Each 800 mg of Rufinamide premix contains 400 mg of Rufinamide.

### Manufacturing Process: Same as Example 1.

### Example 8

Tablet compositions comprising Rufinamide premix prepared by wet granulation method:

| Ingredient | mg/unit |
|---|---|
| Intra-granular ingredients | |
| Rufinamide premix^{$} | 1000.00 |
| Lactose monohydrate | 170.00 |
| Crospovidone | 60.00 |
| Colloidal silicon dioxide | 20.00 |
| Wet granulation | |
| Sodium lauryl sulphate | 20.00 |
| Purified water | q.s. |
| Extra-granular ingredients | |
| Crospovidone | 50.00 |
| Colloidal silocon dioxide | 20.00 |
| Lubrication | |
| Magnesium stearate | 10.00 |
| Film coating | |
| Opadry ® pink | 27.00 |
| Purified water | q.s |
| Film coated tablet weight | 1377.00 |

^{$}Each 1000 mg of Rufinamide premix contains 400 mg of Rufinamide.

### Manufacturing Process:

1. Intra-granular ingredients were sifted through mesh #30 sieve and dry blended for 10 minutes,
2. granulating fluid was prepared using sodium lauryl sulphate and purified water,
3. blend of step 1 was granulated using granulating fluid of step 2, followed by drying and sifting to get desired granules,
4. extra-granular ingredients were sifted through mesh #40 sieve,
5. granules of step 3 were blended with sifted materials of step 4,
6. magnesium stearate was sifted through mesh #60 sieve,
7. granules of step 5 were lubricated with magnesium stearate of step 6,
8. lubricated granules of step 7 were compressed into tablets,
9. tablets of step 8 were film coated using an Opadry® pink dispersion.

1. A pharmaceutical composition comprising
i) a rufinamide premix,
ii) crospovidone as a superdisintegrant and
iii) one or more pharmaceutically acceptable excipients.

2. The pharmaceutical composition according to claim 1, wherein said rufinamide premix comprises a solid dispersion of rufinamide along with a pharmaceutically acceptable carrier selected from hydroxypropyl methylcellulose; copovidone; sorbitan monolaurate; ethyl cellulose; polyethylene glycol; a graft copolymer comprised of polyethylene glycol, polyvinylcaprolactam and polyvinylacetate; and combinations thereof.

3. The pharmaceutical composition according to claim 1, wherein pharmaceutically acceptable excipient is selected from a diluent, a binder, a surfactant, a glidant, a lubricant, and a combinations thereof.

4. The pharmaceutical composition according to claim 1, wherein the composition is prepared by a wet granulation process.

5. A pharmaceutical tablet composition comprising
i) a rufinamide premix,
ii) crospovidone as a superdisintegrant, and
iii) one or more pharmaceutically acceptable excipients selected from lactose, sodium lauryl sulphate, colloidal silicon dioxide, and magnesium stearate.

6. The pharmaceutical composition according to claim 1, wherein the crospovidone is present either extragranularly, or intragranularly or both.

7. The pharmaceutical composition according to claim 1, wherein said rufinamide premix is present in an amount of 50 to 90%, and crospovidone is present in an amount of 1 to 15% by weight relative to the total weight of the composition.

8. A process for preparing compositions of rufinamide premix by wet granulation comprising:
i) sifting and blending a rufinamide premix optionally with one or more pharmaceutically acceptable excipients to form a dry blend,
ii) granulating the dry blend of step (i), using a solvent or binder solution containing sodium lauryl sulphate, followed by drying and sizing to provide granules,
iii) blending the granules of step (ii) with at least one pharmaceutically acceptable excipient,
iv) lubricating the granules of step (iii), and compressing the lubricated granules into tablets.

9. The process according to claim 8, wherein the tablets further comprise crospovidone either extragranularly, intragranularly, or both.

10. A method of treating seizures associated with Lennox-Gastaut syndrome by administering to a subject in need thereof the rufinamide premix of claim 1.

11. The pharmaceutical tablet composition according to claim 5, wherein said rufinamide premix is present in an amount of 50 to 90% by weight, and the crospovidone is present in an amount of 1 to 15% by weight, relative to the total weight of the composition.

12. The pharmaceutical tablet composition according to claim 11, wherein the crospovidone is present in an amount of 3 to 12% by weight relative to the total weight of the composition.

13. The pharmaceutical tablet composition according to claim 5, wherein the crospovidone is present either extragranularly, intragranularly or both.

14. The pharmaceutical composition according to claim 7, wherein the crospovidone is present in an amount of 3 to 12% by weight relative to the total weight of the composition.
